(19) United States
(12) Patent Application Publication
(10) Pub. No.: US 2011/0059050 A1
(43) Pub. Date: Mar. 10, 2011

(54) METHODS AND COMPOSITIONS RELATING TO STEM CELL TRANSPLANTATION

(76) Inventors: Kyle Cetrulo, Amesbury, MA (US); Curtis Cetrulo, Amesbury, MA (US)

(21) Appl. No.: 12/161,783

(22) PCT Filed: Jan. 26, 2007
(86) PCT No.: PCT/US2007/002244

§ 371 Date: Nov. 29, 2010

Related U.S. Application Data
(60) Provisional application No. 60/762,814, filed on Jan. 27, 2006.

(51) Int. Cl. A61K 35/12; A61P 35/00

(52) U.S. Cl. 424/93.7

(57) ABSTRACT

The invention relates to methods and compositions for stem cell transplantation. Aspects of the invention relate to administering hematopoietic stem cells and mesemchymal cells to a patient.

### RELATED APPLICATION

This application claims the benefit under 35 U.S.C. §119(e) of U.S. provisional patent application Ser. No. 60/762,814, filed Jan. 27, 2006, and the contents of which are incorporated herein by reference in their entirety.

### FIELD OF THE INVENTION

The invention relates to compositions of stem cells and stem cell transplantation.

### BACKGROUND OF THE INVENTION

During mammalian embryogenesis, the placenta is the first organ formed. The placenta is formed from the rudimentary allantois. The allantois is first seen in embryologic life at day E7.5 and forms the umbilical cord and mesodermal components of the fetal placenta. By day E12.5 the placenta is formed. The placenta is a source of hematopoietic stem cells (HSCs) that can self-renew and provide multi-lineage, long-term reconstitution in adult recipients. The ontogeny or development of hematopoietic stem cells (HSCs) begins soon after gastrulation (E7.5). The first site of hematopoiesis is the extraembryonic yolk sac, soon followed by the intraembryonic aorta-gonad-mesonephros (AGM) region. The AGM region generates the adult hematopoietic system and HSCs. The onset of HSCs activity coincides with AGM and yolk sac and precedes fetal liver and circulating blood. From the AGM region there is a migration of a common unrestricted precursor to the fetal liver through the allantois. HSC from human umbilical cord blood (or cord blood as used interchangeably herein) were first described in 1974.

HSC transplant is the only curative treatment for a number of malignant and non-malignant diseases or in situations where the blood cells have been damaged or become abnormal. The use of HSC transplant has been steadily increasing, both because of its demonstrated effectiveness in selected diseases and because of increasing availability of donors. The first transplant of cord blood HSC into a patient with Fanconi's anemia was reported by Gluckman in 1989 in the New England Journal of Medicine. As of March 2004, there have been over 6000 transplants using cord blood HSCs.

Despite significant advances in the field of transplant, hurdles remain in improving the outcome of hematopoietic stem cell transplant. One major hurdle to be overcome is the need for improved homing and engraftment of the transplanted cells. For example, a major drawback of cord blood HSC transplantation is the delayed engraftment due to the restricted number of cells in a cord blood sample. The nucleated cell dose is the most important determinant along with HLA typing to determine the success and speed of engraftment. A nucleated cell dose of <2.5×10⁷ shows poor success and engraftment. In one study, only 25 percent of cord blood samples collected met this critical level. (Kogler, G Eurocord/Netcord Bank Germany, Klin Podiati 1999; 211:224-232). For this reason, different approaches to the use of cord blood transplantation have been suggested including using double unit transplants and expansion of stem cells prior to transplant. For example, Wagner and Barker have successfully used two cord blood units to transplant adult patients. (Barker J N et al. Transplantation of two partially HLA-matched umbilical cord blood units to enhance engraftment in adults with hematologic malignancy. Blood 2005; 105:1343-1347). Another problem of HSC transplant generally is rejection such as graft versus host disease (GVHD).

There exists a need for more efficient stem cell transplantation methods.

### SUMMARY OF THE INVENTION

This invention relates broadly to improved methods for stem cell transplantation. In important aspects of the invention, the stem cells are HSC. The invention is premised on the discovery that when HSC are combined with umbilical cord matrix (UCM) cells (i.e., UCMCs or cells obtained from Wharton's Jelly) the transplantation of such HSCs is greatly enhanced including more rapid engraftment, longer engraftment and a better therapeutic result. Additionally, smaller numbers of HSCs obtained from cord blood, in combination with UCMC, may now be used.

The combination of UCMCs and HSCs creates a unique cocktail for stem cell transplantation. Such transplantation is useful in reconstitution of the hematopoietic lineages. Due to the plasticity of the HSC, these methods will also be useful in regenerative medicine and gene therapy for treating genetic disorders, cardiac disease, neurological disorders such as stroke, Alzheimer's disease, Parkinson's disease and spinal cord injuries, endocrine disease such as diabetes, and orthopedic problems. There are now more than 70 diseases that have responded to HSC transplantation and the invention can be used in the treatment of any of these. Accordingly, the invention provides a non-controversial source of stem cells such as HSCs.

Thus, in one aspect, the invention provides a method for transplanting cells in subject in need thereof comprising administering to the subject stem cells such as HSC and mesenchymal cells in numbers sufficient to populate one or more lineages in the subject.

The HSC may be obtained from a number of sources including but not limited to cord blood, bone marrow and mobilized peripheral blood.

The mesenchymal cells may be mesenchymal stem cells. They may be obtained from UCM (i.e., Wharton's Jelly). In some embodiments, they have a phenotype of CD105+ (SH-2+), CD73+ (SH-3+ and SH-4+), CD34−, CD45−, and/or CD14−.

In one embodiment, the HSC and/or the mesenchymal cells are isolated, and/or partially purified from their source.

In one embodiment, the HSC and mesenchymal cells are administered intravenously.

In another embodiment, the HSC and mesenchymal cells are administered locally including but not limited to within a tissue or a tumor.

In one embodiment, the subject has a disorder treatable by hematopoietic stem cell transplantation. As described in greater detail herein the disorder may be a hematopoietic deficiency or malignancy, but it is not so limited.

In one embodiment, the subject has a blood or hematopoietic disorder which may be but is not limited to myelodysplasia, aplastic anemia, Fanconi's anemia, Sickle cell disease, Diamond Blackfan anemia, Schachman Diamond disorder, and thalassemia. It may alternatively be a leukemia, such as acute or chronic leukemia. The acute leukemia may be selected from the group consisting of acute lymphocytic leukemia (ALL) and acute myelogenous (myeloid) leukemia (AML). The chronic leukemia may be selected from the group consisting of chronic lymphocytic leukemia (CLL), B-cell chronic lymphocytic leukemia, chronic myelogenous leukemia (CML), juvenile chronic myelogenous leukemia (JCML), and juvenile myelomonocytic leukemia (JMML).

In another embodiment, the subject has a lymphoma such as but not limited to T cell lymphoma, B cell lymphoma such as Hodgkin's lymphoma and non-Hodgkin's lymphoma.

In another embodiment, the subject has an immunodeficiency such as but not limited to severe combined immunodeficiency disease (SCID).

The subject may also have a solid cancer. The solid cancer may be selected from the group consisting of breast cancer, renal cell (kidney) cancer, ovarian cancer, pancreatic cancer, small-cell lung cancer, melanoma, neuroblastoma, and pediatric sarcoma.

The subject may have a metabolic disorder such as but not limited to amyloidosis and diffuse progressive systemic sclerosis.

The subject may have a genetic disorder such as but not limited to alpha-L-iduronidase deficiency syndrome (Hurler's syndrome or disease), osteopetrosis, Gaucher's disease, globoid cell leukodystrophy, and adrenoleukodystrophy.

In an important embodiment, the method results in more rapid engraftment compared to transplantation of the hematopoietic stem cells in the absence of the mesenchymal cells.

In another important embodiment, the method results in higher engraftment levels compared to transplantation of the hematopoietic stem cells in the absence of the mesenchymal cells.

In still another important embodiment, the number of hematopoietic stem cells administered is below a number required to repopulate at least one lineage in the subject in the absence of the mesenchymal cells.

In one embodiment, the hematopoietic stem cells and mesenchymal cells are combined before administration to the subject.

In one embodiment, the mesenchymal cells are administered before, during and after the hematopoietic stem cells. In another embodiment, the mesenchymal cells are administered during and after the hematopoietic stem cells. In still another embodiment, the mesenchymal cells are administered after the hematopoietic stem cells. The mesenchymal cells may be administered on a regular basis or schedule including but not limited to daily, weekly, biweekly, or monthly.

In still another embodiment, the method further comprises administering a therapeutic agent to the subject, such as but not limited to an immunosuppressive agent. The immunosuppressive agent may be a steroid. The steroid may be a corticosteroid which may be selected from the group consisting of prednisone and methylprednisone. The immunosuppressive agent may be a calcineurine inhibitor such as cyclosporine and tacrolimus, although it is not so limited. The immunosuppressive agent may otherwise be selected from the group consisting of mycophenolate mofetil (MMF), azathioprine and sirolimus. It may be a monoclonal antibody including but not limited to anti-CD3 antibody (Muromonab-CD3; Orthoclone OKT3®) and anti-IL-2R alpha chain (p55). It may be a polyclonal antibody including but not limited to anti-thymocyte globulin-equine (Atgam®) and anti-thymocyte globulin-rabbit (RATG Thymoglobulin®). The immunosuppressive agent may otherwise be selected from the group consisting of sulfasalazine, FK-506, methoxsalen, and thalidomide.

In another aspect, the invention provides a method for improving the outcome of a hematopoietic stem cell transplant in a subject comprising administering to a subject in need thereof hematopoietic stem cells and mesenchymal cells in an amount sufficient to improve the outcome of the hematopoietic stem cell transplant in the subject as compared to the outcome in the absence of the mesenchymal cells.

According to another aspect, the invention provides pharmaceutical compositions comprising isolated hematopoietic stem cells and isolated mesenchymal cells formulated in a pharmaceutically acceptable carrier. These pharmaceutical compositions may be useful in therapy. For example, the pharmaceutical compositions of the invention may be useful for treating a subject having a disorder that benefits from populating at least one cell lineage.

In some embodiments, the hematopoietic stem cells are cord blood stem cells. In certain embodiments, the mesenchymal cells are mesenchymal stem cells. The mesenchymal cells may be umbilical cord matrix cells.

Accordingly, pharmaceutical compositions of the invention may be useful for treating one or more of a number of diseases, including but not limited to: myelodysplasia, aplastic anemia, Fanconi's anemia, Sickle cell disease, Diamond Blackfan anemia, Schachman Diamond disorder, thalassemia, acute lymphocytic leukemia (ALL); acute myelogenous (myeloid) leukemia (AML), chronic lymphocytic leukemia (CLL), B-cell chronic lymphocytic leukemia, chronic myelogenous leukemia (CML), juvenile chronic myelogenous leukemia (JCML), juvenile myelomonocytic leukemia (JMML), T cell lymphoma, B cell lymphoma, Hodgkin's lymphoma, non-Hodgkin's lymphoma, severe combined immunodeficiency disease (SCID), breast cancer, renal cell (kidney) cancer, ovarian cancer, pancreatic cancer, small-cell lung cancer, melanoma, neuroblastoma, pediatric sarcoma, amyloidosis, diffuse progressive systemic sclerosis, alpha-L-iduronidase deficiency syndrome (Hurler's syndrome or disease), osteopetrosis, Gaucher's disease, globoid cell leukodystrophy, and adrenoleukodystrophy.

In yet another aspect, the invention provides a use of isolated hematopoietic stem cells and isolated mesenchymal cells for the manufacture of a composition or pharmaceutical preparation for treating a disorder that benefits from populating at least one cell lineage.

In some embodiments, the hematopoietic stem cells are cord blood stem cells. In certain embodiments, the mesenchymal cells are mesenchymal stem cells. The mesenchymal cells may be umbilical cord matrix cells.

Accordingly, use of isolated hematopoietic stem cells and isolated mesenchymal cells described herein may relate to the manufacture of a composition or pharmaceutical preparation for treating one or more of a number of disorders, including but not limited to: myelodysplasia, aplastic anemia, Fanconi's anemia, Sickle cell disease, Diamond Blackfan anemia, Schachman Diamond disorder, thalassemia, acute lymphocytic leukemia (ALL), acute myelogenous (myeloid) leukemia (AML), chronic lymphocytic leukemia (CLL), B-cell chronic lymphocytic leukemia, chronic myelogenous leukemia (CML), juvenile chronic myelogenous leukemia (JCML), juvenile myelomonocytic leukemia (JMML), T cell lymphoma, B cell lymphoma, Hodgkin's lymphoma, non-Hodgkin's lymphoma, severe combined immunodeficiency disease (SCID), breast cancer, renal cell (kidney) cancer, ovarian cancer, pancreatic cancer, small-cell lung cancer, melanoma, neuroblastoma, pediatric sarcoma, amyloidosis, diffuse progressive systemic sclerosis, alpha-L-iduronidase deficiency syndrome (Hurler's syndrome or disease), osteopetrosis, Gaucher's disease, globoid cell leukodystrophy, and adrenoleukodystrophy.

It is to be understood that many if not all of the foregoing embodiments recited for one aspect of the invention relate equivalently to other aspects of the invention. Various aspects and embodiments of the invention will be described in greater detail below. The invention intends to embrace various combinations of embodiments, unless otherwise stated.

### DETAILED DESCRIPTION OF THE INVENTION

The invention relates broadly to methods and compositions for improving stem cell transplantations. According to the invention, stem cell transplantation outcome can be improved by administering to a subject a combination of the stem cells to be transplanted along with a population of mesenchymal cells.

### Hematopoietic Stem Cells

The stem cells are preferably hematopoietic stem cells, although they are not so limited. Hematopoietic stem cells can be obtained from umbilical cord blood, bone marrow, mobilized peripheral blood, and in some instances differentiated embryonic stem cells. Other stem cells that can be used in the methods of the invention include kidney stem cells, skin stem cells, neural stem cells and the like. Other sources of stem cells include placenta, amniotic fluid, umbilical vein, and decidua.

Cord blood from singleton gestations, or cord blood collected in utero (as compared to ex utero), cord blood from an early cord clamping procedure and harvest (i.e., within 5 minutes of delivery), cord blood from longer cords, and cord blood from increased birth weight and placental weight deliveries are sometimes preferred.

Hematopoietic stem cells have been characterized in the art. The cells in the human generally have minimally a CD34+CD33− phenotype. They can be harvested from bone marrow, cord blood or peripheral blood using affinity columns, magnetic beads, fluorescence activated cell sorting (FACS), some combination thereof, and the like.

These cells have the ability to repopulate one or more hematopoietic lineages upon transplantation. Preferably, these cells repopulate more than one lineage, and even more preferably, all lineages. Repopulation or population of lineages as used herein refers to the differentiation of the stem cell into one or more lineages such that progeny of the stem cell contribute to the make up of that lineage in the subject. It does not however require that the entire lineage compartment derive from the transplanted cells, however in some instances this may occur.

Recently, plasticity in the differentiative potential of HSC has been reported. Plasticity refers to the ability of the HSC to “de-differentiate” and actually produce cells that are non-hematopoietic in nature. Accordingly, the invention embraces the use of HSC transplantation for the repopulation or population of lineages, tissues and/or organs that are not hematopoietic in nature.

### Mesenchymal Cells

The mesenchymal cells are preferably mesenchymal stem cells. Mesenchymal stem cells can be obtained from bone marrow or umbilical cord matrix (i.e., Wharton's Jelly), among other tissues. The UCM represent a rich source of primitive multipotent mesenchymal cells with important characteristics including high telomerase activity responsible for their self renewal capacity, and immortality. Harvest of mesenchymal stem cells from UCM generally at delivery has been described previously. (See Weiss et al. Stem Cells, 2005, Eprint; Ma et al. Chin Med J., 2005, 118:1987-93; Wang et al. 2004 Stem Cells 22:1330-1337; Covas et al. 2003 Brat J Med Biol Res. 36:1179-83; Mitchell et al. 2003 Stem Cells 21:50-60.) These cells are easily accessible compared to bone marrow MSC, are more abundant than in cord blood and they lack the ethical considerations of ES cells.

Mesenchymal stem cells can differentiate into neuronal cells, adipocytes, chondroblasts, osteoblasts, myocytes, cardiac tissue, and other endothelial and epithelial cells. (See Wang, Stem Cells 2004; 22(7); 1330-7; McElreavey; 1991 Biochem Soc Trans (1); 29s; Takechi, Placenta 1993 March/April; 14 (2); 235-45; Takechi, 1993; Kobayashi; Early Human Development; 1998; July 10; 51 (3); 223-33; Yen; Stem Cells; 2005; 23 (1) 3-9.) They are therefore candidates for cell-based therapies. In contrast to ES cells, but like cord blood cells, UCM cells display more immune tolerance and have been shown not to form tumors when injected into immune-compromised mice.

These cells proliferate in culture and express markers found in other stem cells. Mesenchymal cells from UCM are positive for CD 10, SH2/CD105, SH3, CD29, CD13, CD44, CD49e, CD51, CD54, CD73, CD90, CD166, and HLA class I and negative for CD33, CD34, CD45, CD56, CD31, CD14, glycophorin A, HLA-DR, CD106, and CD49d. The adherent cell population which contains the mesenchymal stem cells is identified based on fibroblastoid phenotype and the expression of mesenchymal markers CD105^{+(SH-2)}, CD73^{+(SH3)} and CD34⁻, CD45−. These cells also express Oct-4 and Nanog. After freezing and thawing UCMs are reportedly negative for MHC class I and II. Another population of mesenchymal cells is the human umbilical cord perivascular cells (HUCPV) which do not present either MHC class I or II. Mesenchymal stem cells from bone marrow have CD 105+ (SH-2+), CD73+ (SH-3+ and SH-4+), CD34−, CD45−, CD14− phenotype.

### Isolated and Purified

The stem cells may be isolated. As used herein, an isolated cell is one that is separated physically from components with which it normally resides, for example in vivo. As an example, an isolated umbilical cord blood stem cell is separated physically from for example other umbilical cord and cord blood components such as umbilical cord matrix (i.e., Wharton's Jelly).

The isolated stem cells may be obtained by fractionating a heterogenous cell population according to one or more markers, including by not limited to cell surface markers. As an example, isolated hematopoietic stem cells can be fractionated from bone marrow, for example, based on expression of CD34 and CD45 and non-expression of CD38. A CD34+, CD45+, CD38− stem cell population is referred to herein as a partially purified population of stem cells.

Mesenchymal stem cells can be fractionated from a heterogenous population including bone marrow and umbilical cord matrix. Such fractionated populations are referred to herein as partially purified populations of mesenchymal cells.

The cells to be transplanted can be cultured and/or expanded in vitro prior to administration to the subject, however, the invention is not so limited.

### Administration Timing

The invention contemplates administration of HSC and mesenchymal cells at the same or different times. The mesenchymal cells can be administered before and/or during and/or after administration of the HSC.

In one embodiment, the mesenchymal cells are administered after the HSC cells. As an example, the HSC can be infused in a subject on day 1, after which the subject may be administered mesenchymal cells one or more times in the days, weeks or months following HSC administration. The mesenchymal cells may be administered regularly for example every day, every week, every two weeks, every month, etc. Alternatively or additionally, they may be administered when the subject appears in need for example when engraftment levels are declining or when the subject is getting infections (which may be indicative of poor engraftment).

### Transplantation

Cell transplantation can be categorized according to the relationship between the recipient and the donor. The major categories are syngeneic, allogeneic, autologous and xenogeneic transplantation. A syngeneic transplant involves a donor and a recipient who are immunologically identical. A transplant between two identical twins is an example of a syngeneic transplant. An allogeneic transplant involves a donor and recipient who are not immunologically identical. An autologous transplant involves the removal and storage of the patient's own stem cells with subsequent reinfusion after the patient receives high-dose myeloablative therapy. The different categories of hematopoietic stem cell transplant are encompassed by this invention.

The invention contemplates the use of HSC from one donor (or perhaps from the recipient) together with mesenchymal cells from the same or a different donor. Accordingly, while in some instances the HSCs and the mesenchymal cells will derive from the same subject, the invention is not so limited.

The transplants of the invention can be used to repopulate part or all of a subject's hematopoietic system. Alternatively or additionally, it may be used to populate another tissue or lineage that is non-hematopoietic in nature. These latter tissue or lineages include but are not limited to neural tissues and lineages.

Hematopoietic stem cell transplant refers to the collection and transplantation of hematopoietic stem cells. The procedure may be carried out, for example, to treat malignancy by allowing the administration of higher doses of myelosuppressive therapy than would otherwise be possible. It may also be carried out to replace an abnormal but nonmalignant hematopoietic system with one from a normal donor.

HSCs may be harvested and/or isolated from for example cord blood, bone marrow and mobilized peripheral blood. Cord blood and bone marrow harvests are known in the art. Preparation of mobilized peripheral blood progenitors can be achieved using growth factors as described herein. Following the administration of certain hematopoietic growth factors, including granulocyte colony stimulating factor (G-CSF) or granulocyte-macrophage colony stimulating factor (GM-CSF), the concentration of hematopoietic progenitor cells in blood, as measured either by colony forming units or expression of the CD34 antigen, increases markedly. Thus, it is possible to harvest hematopoietic stern cells from the peripheral blood for transplant. Donors are typically treated for four or five days with the hematopoietic growth factor, following which stem cells are collected in one or two 4-h pheresis sessions. In the autologous setting, transplant of >2.5×10⁶ CD34⁺ cells per kilogram is generally used to engraft a recipient. Peripheral blood stem cells are collected by leukopheresis.

Any of these HSC populations may be further processed prior to administration to a subject. The nature of processing will vary and may include removal of red cells (for example by density separation) to prevent hemolysis in ABO-incompatible transplants, the removal of donor T cells to prevent GVHD, or removal of possible contaminating tumor cells in autologous transplant.

### Engraftment

Although not intending to be bound by any particular mechanism or theory, the methods and compositions of the invention improve engraftment and/or homing of the transplanted stem cells, potentially leading to an increase in the number of stem cells in the subject.

As used herein, the term “improving” refers to achieving a medically better benefit and/or outcome than would be achieved in the absence of combining the transplanted stem cells with the mesenchymal cells described herein.

By way of example, measures of improvement of the outcome of a hematopoietic stem cell transplant include increased hematopoietic stem cell engraftment, increased homing, increased numbers of stem cells, and reduced graft versus host disease (GVHD).

As used herein, “engraftment” is a re-population of part or all of a recipient's cell type, organ, tissue, or system (such as the hematopoietic system) with cells produced by the transplanted stem cells. In some instances, engraftment occurs 1, 2, 3, 4, 5, 6, 7, 8, 9, 10, 11, 12, or more days faster than it would have in the absence of the mesenchymal cells. More rapid engraftment reduces the likelihood of infection and anemia in the transplant recipient.

In the case of hematopoietic stem cell transplants, peripheral blood counts usually reach their nadir several days to a week post-transplant, then cells produced by the transplanted stem cells begin to appear in the peripheral blood. The rate of recovery depends on the source of stem cells, the use of post-transplant growth factors, and the form of GVHD prophylaxis employed. Use of myeloid growth factor (G-CFS or GM-CSF) post-transplant can further accelerate recovery, while use of methotrexate to prevent GVHD may delay engraftment.

Engraftment can be measured using fluorescence in situ hybridization of sex chromosomes if donor and recipient are sex-mismatched, HLA-typing if HLA-mismatched, or restriction fragment length polymorphism analysis if sex- and HLA-matched.

Engraftment may also be measured by Short Tandem Repeat (STR) analysis/testing. STR analysis is also useful for following the subject's course during treatment. STR analysis is useful for testing allogeneic or unrelated bone marrow transplant from a non-identical twin donor. Donor and recipient specific DNA fragment patterns are utilized to distinguish the cell populations. One molecular marker will be used to detect the engraftment of normal donor cells in the recipient post-transplant.

DNA isolated from the peripheral blood lymphocytes of the patient and donor is characterized with several variable DNA markers from different chromosomes prior to transplant. This analysis will characterize the specific patterns demonstrated at each marker and indicate which marker may be most suitable for distinguishing donor from recipient. This marker will be used to subsequently assess the status of engraftment. The DNA markers are used in fluorescent multiplex PCR assays to identify the DNA fragment patterns of both recipient and donor.

One example of such a molecular DNA analysis test is provided by BJC Health systems (St. Louis, Mo.). In this test 5 DNA markers (see below) are used. In some tests less than 5 DNA markers may be used. In some tests, 2 DNA markers may be used. In other tests, more than 5 markers may be used. The separate markers vary in the number of copies of tandem 4 bp sequence repeats (Hammond, et al.; Am. J. Hum. Genet. 55: 175-189. 1994). The alleles range in size and heterozygosity as indicated below. In the general population, only identical twins demonstrate identical DNA profiles at all of these STR loci. In related family members, one or more of these markers may demonstrate high incidence of non-identity in the hematopoietic stem cell transplant setting (Scharf, et al.; Blood 85: 1954-63, 1995).

| | | | Hetero- | |
|---|---|---|---|---|
| | | | zygosity | Fragment size |
| Marker | Repeat | Chromosome assignment | (%) | range (bp) |
| THO1 | AATG | Tyrosine hydroxylase | 77 | 179-203 |
| | | 11p15.5 | | |
| TPOX | AATG | Thyroid peroxidase | 75 | 224-252 |
| | | 2p23-2pter | | |
| CSFPO1 | AGAT | CSF1 c-fms | 74 | 295-327 |
| | | 5q33.3-34 | | |
| VWA | AGAT | von Willebrand factor | 65 | 139-167 |
| | | 12p12-pter | | |
| F13A01 | AAAG | Coagulation factor XIII | 73 | 283-331 |
| | | 6p24-25 | | |
| Amelo- | NA | X p22.1-22.3 and Y | — | X = 212 bp; |
| genin* | | | | Y = 218 bp |

A recipient and donor may be a) identical for both alleles A and B (non-informative), b) have one common allele and one unique allele each (informative), or c) have no alleles in common (informative). The recipient and donor patterns must be different from each other (informative) to assess engraftment of donor specific cells. When more than one of the STR markers has an informative pattern, the one that demonstrates the most clear differences in size or mobility is selected to follow engraftment status post-transplant. (*Amelogenin distinguishes X and Y chromosomes and is only used when a) the recipient and donor are not of the same gender and b) all other STR markers are not informative.) Specimen requirements for this test include peripheral blood or bone marrow specimens (3-5 ml or 1-3 ml, respectively) collected in sodium EDTA tubes.

Other tests for measuring hematopoietic stem cell homing and engraftment include CXCL12-induced migration as described by Christopherson et al. (Science 305, 1000-1003, 2004) and assessment of transplanted hematopoietic cells to bone marrow using congenic C57B1/6 (CD45.2⁺) and BoyJ (CD45.1⁺) cells as described by Christopherson et al. (Science 305, 1000-1003, 2004) and Harrison (Blood 55:77-81, 1980).

Modifications of the above tests and methods are within the knowledge and skill of those of ordinary skill in the art and are contemplated by this invention.

Engraftment may be in the short term period after transplant (short term engraftment), in the long term period after transplant (long term engraftment), or both. The short term period after transplant is the period of one day until up to six months after transplant. The long term period after transplant is the period beyond six months after transplant.

Another measure of improvement of the outcome of a hematopoietic stem cell transplant is a reduced GVDH. Methods of assessing GVHD are known to those of ordinary skill in the art. These methods include clinical evaluation of the recipient for signs and symptoms of GVHD. Improving the outcome of a hematopoietic stem cell transplant therefore may include reducing the incidence or severity of GVHD and its associated symptoms.

In acute cases GVHD symptoms include pruritic or painful rash, fever, generalized erythroderma, and desquamation, diarrhea, intestinal bleeding, cramping abdominal pain, anorexia, dyspepsia, ileus, infectious and noninfectious pneumonia and sterile effusions. A hemolytic-uremic syndrome (thrombotic microangiopathy) may occur. Laboratory findings include elevation of bilirubin, alanine aminotransferase (ALT), aspartate aminotransferase (AST), alkaline phosphatase, thrombocytopenia, and anemia.

Chronic GVHD is viewed as an extension of acute GVHD. However, it also may occur de novo in patients who never showed clinical evidence of acute GVHD, or it may emerge following a quiescent interval after acute GVHD resolution. Ocular manifestations include burning, irritation, photophobia, and pain from lack of tear secretion. Oral and gastrointestinal manifestations include dryness, sensitivity to acidic or spicy foods, dysphagia (difficulty in swallowing), odynophagia (pain upon swallowing), and insidious weight loss. Pulmonary manifestations include obstructive lung disease with symptoms of wheezing, dyspnea. Chronic cough that usually is nonresponsive to bronchodilator therapy is a clinical feature of chronic GVHD. Neuromuscular manifestations include weakness, neuropathic pain, and muscle cramps.

Physical findings of GVHD include findings and or manifestations in the skin, liver (hepatic), eye (ocular), oral pulmonary, neuromuscular as well as in other organs.

Skin findings include lesions that are red to violaceous in color and typically first appear on the palms of the hands and the soles of the feet, the cheeks, neck, ears, and upper trunk. It can progress to involve the whole body. In severe cases, bullae may be observed, and vesicular formation may occur. Chronic GVHD can lead to lichenoid skin lesions or sclerodermatous thickening of the skin sometimes causing contractures and limitation of joint mobility.

Hepatic manifestations include hyperbilirubinemia which can manifest as jaundice and can cause pruritus and lead to excoriations from the patient's scratching. Other rare hepatic manifestations include portal hypertension and cirrhosis. Ocular manifestations include hemorrhagic conjunctivitis, pseudomembrane formation, and lagophthalmos. Keratoconjunctivitis sicca is common. Punctate keratopathy (minimal or severe erosions of the cornea) may also be present. Oral manifestations include atrophy of oral mucosa, erythema, and lichenoid lesions of the buccal and labial mucosae all of which correlate significantly with the development of chronic GVHD. Pulmonary manifestations include bronchiolitis obliterans. Gastrointestinal manifestations include diffuse abdominal tenderness with hyperactive bowel sounds which may accompany a secretory diarrhea. In severe ileus, the abdomen is silent and appears distended. Neuromuscular findings include autoimmune phenomenon of myasthenia gravis or polymyositis. Other findings include vaginitis and vaginal strictures. Autoimmune thrombocytopenia and anemia may also be observed.

Chronic GVHD has manifestations similar to those of systemic progressive sclerosis, systemic lupus erythematosus, lichen planus, Sjögren syndrome, eosinophilic fasciitis, rheumatoid arthritis, and primary biliary cirrhosis.

Signs, symptoms and abnormal laboratory findings of GVHD are observed less frequently than would be observed in the absence of the described therapy or intervention.

In vitro tests are also available to assess GVHD. Examples of such tests are described by Sviland et al. (Bone Marrow Transplant 5: 105-109, 1990) and by Vogelsang et al. (NEJM, 313:645-650, 1985).

### Subjects

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops), provided that it would benefit from the methods provided herein. Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. In all embodiments human subjects are preferred. Human subjects can be subjects at any age, including adults, juveniles, infants and fetuses in utero.

### Disorders

In some embodiments, the subject has been myeloablated, either intentionally or accidentally, for example via chemotherapy, radiotherapy, or accidental radiation exposure. Subjects myeloablated via chemotherapy and/or radiotherapy include subjects having cancer, including but not limited to leukemia, lymphoma, and breast cancer.

The subject in need of transplantation is a subject having a disorder to condition that can be treated by stem cell transplantation. The disorder may be a hematopoietic disorder or a non-hematopoietic disorder.

Examples of hematopoietic disorders include myelodysplasia, aplastic anemia, Fanconi's anemia, paroxysmal nocturnal hemoglobinuria, Sickle cell disease, Diamond Blackfan anemia, Schachman Diamond disorder, Kostmann's syndrome, chronic granulomatous disease, leukocyte adhesion deficiency, and thalassemia including thalassemia major. In one embodiment the subject is preferably transplanted before developing hepatomegaly or portal fibrosis.

In some embodiments the hematopoietic disorders is leukemia. The leukemia may be an acute leukemia. Examples of acute leukemias include acute lymphocytic leukemia (ALL), acute myelogenous (myeloid) leukemia (AML), and adult lymphoblastic leukemia. The leukemia may be a chronic leukemia. Examples of chronic leukemias include chronic lymphocytic leukemia (CLL), B-cell chronic lymphocytic leukemia, chronic myelogenous leukemia (CML), juvenile chronic myelogenous leukemia (CML), and juvenile myelomonocytic leukemia (JMML).

In some embodiments the subject has severe acquired autoimmune disorder.

In some embodiments the subject has an immunodeficiency such as but not limited to severe combined immunodeficiency disease (SCID), Wiskott-Aldrich syndrome and Chédiak-Higashi syndrome.

In other embodiments the subject has a lymphoma such as but not limited to Hodgkin's lymphoma and non-Hodgkin's lymphoma (NHL). Hodgkin's lymphoma includes advanced Hodgkin's lymphoma (poor risk, relapsed and refractory). Examples of NHL include diffuse large cell NHL, follicular NHL, and low grade NHL.

In some embodiments the subject has a metabolic disorder. Examples of metabolic disorders include but are not limited to amyloidosis and diffuse progressive systemic sclerosis.

In other embodiments the subject has a genetic disorder. Examples of genetic disorders include alpha-L-iduronidase deficiency syndrome (Hurler's syndrome or disease), osteopetrosis, Gaucher's disease, globoid cell leukodystrophy, Hunter's syndrome, infantile metachromatic leukodystrophy, and adrenoleukodystrophy.

In one embodiment the subject has infantile malignant osteoporosis.

Other disorders include acute leukemias such as ALL and AML; chronic leukemias such as CLL and CML; lymphoproliferative disorders such as NHL, Hodgkin's Disease, and prolymphocytic leukemia; myelodysplastic syndromes such as refractory anemia; neuroblastoma; inherited erythrocyte abnormalities such as B-thalassemia major, pure red cell aplasia, and sickle cell disease; liposomal storage diseases such as mucopolysaccaridoses, Hurler's syndrome, Hunter's syndrome, adrenoleukodystrophy, Gaucher's disease, and Niemann-Pick disease; congential immune disorders such as SCID, Wiskott-Aldrich syndrome and X-linked lymphoproliferative disorder; plasma cell disorders such as multiple myeloma, plasma cell leukemia and Waldenstrom's macroglobulenemia; other inherited diseases such as Lesch-Nyhan syndrome and osteopetrosis; and phagocyte disorders such as Chediak-Higashi syndrome and chronic granulomatous disease.

The invention may also be used to treat non-hematopoietic disorders such as but not limited to cardiac disorders including myocardial infarction and congestive heart failure, gastro-intestinal disorders, endocrine disorders, liver disorders, neurological disorders, and stroke.

The invention further contemplates use of these methods and compositions for more rapid growth and population of cartilage and/or bone.

### Administration

The cells may be administered to the subject via any medically acceptable and appropriate route. Administration may be systemic such as intravenous administration or it may be local such as intra-tissue or intra-tumor administration. An example of local administration includes administration directly into CNS tissues such brain. If done by intravenous routes, it is expected that the HSCs will be administered by infusion over a period of time for example over a number of hours.

### Therapeutic Agents

The invention contemplates administration of therapeutic agents to the subject undergoing stem cell transplantation. Examples of such therapeutic agents include immunosuppressive agents and growth factors. Examples of immunosuppressive agents include but are not limited to steroids, calcineurine inhibitors, monoclonal antibodies and polyclonal antibodies. The steroid may be a corticosteroid. Examples of corticosteroids include but are not limited to prednisone and methylprednisone. Examples of calcineurine inhibitors include but are not limited to cyclosporine and tacrolimus. Examples of monoclonal antibodies include but are not limited to anti-CD3 antibody (Muromonab-CD3; Orthoclone OKT3®) and anti-IL-2R alpha chain (p55). Examples of polyclonal antibodies include but are not limited to anti-thymocyte globulin-equine (Atgam®) and anti-thymocyte globulin-rabbit (RATG Thymoglobulin®). Other examples of immunosuppressive agents include sulfasalazine, FK-506, methoxsalen, thalidomide, mycophenolate mofetil (MMF), azathioprine and sirolimus.

### Effective Amounts

The cell populations are administered in numbers that are sufficient for improving engraftment in a subject. This number may vary with the subject's age, condition, and sex, as well as the extent of the disease in the subject and can be determined by one of skill in the art.

As an example, 6×10⁶ CD34+ cells on average are obtained from a single cord blood sample and on average 5×10⁴ CD34+ cells per kilogram patient body weight are required for transplant.

In some embodiments, the number of HSCs (for example 34+ cells) to be administered can be reduced by at least 5%, 10%, 15%, 20%, 25%, 30%, 35%, 40%, 45%, or at least 50% of the numbers that would be required for engraftment in the absence of the mesenchymal cells. As an example, in some embodiments, the number of nucleated HSC is less than 5×10⁶, less than 1×10⁷, less than 2×10⁷, less than 5×10⁷, less than 1×10⁸, less than 2×10⁸. The total number of cells to be administered will depend upon the subject's weight; however, it is expected that the invention will provide the benefit of lower cell numbers required for each transplant.

### Pharmaceutical Preparations

The cells are formulated as pharmaceutical compositions or preparations. A pharmaceutical preparation is a composition suitable for administration to a subject. Such preparations are usually sterile and prepared according to GMP standards, particularly if they are to be used in human subjects. In general, a pharmaceutical composition or preparation comprises the cells and a pharmaceutically-acceptable carrier, wherein the cells are generally provided in effective numbers. As used herein, a pharmaceutically-acceptable carrier means a non-toxic material that does not interfere with the effectiveness of the biological activity of the cells and other agents of the invention.

Pharmaceutically-acceptable carriers include diluents, fillers, salts, buffers, stabilizers, solubilizers and other materials which are well-known in the art. Exemplary pharmaceutically-acceptable carriers for peptides in particular are described in U.S. Pat. No. 5,211,657. Such preparations may routinely contain salt, buffering agents, preservatives, compatible carriers, and optionally other therapeutic or prophylactic agents. When used in medicine, the salts should be pharmaceutically acceptable, but non-pharmaceutically-acceptable salts may conveniently be used to prepare pharmaceutically-acceptable salts thereof and are not excluded from the scope of the invention. Such pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Kits

The invention further provides kits that comprise reagents necessary to isolate the transplanted cells from one or more sources. For example, the kit may contain the reagents necessary to isolate mesenchymal cells from umbilical cord matrix and/or the reagents necessary to isolate hematopoietic stem cells from umbilical cord blood. These reagents include affinity columns, antibodies, wash solutions and the like. The kit may further include instructions for use of the kit components.

### In Vitro Applications

The invention further relates to the use of mesenchymal cells, preferably UCMC in the growth of HSC and other progenitors in vitro. The UCMC can be used as a supportive layer of cells upon which HSC, including cord blood HSC grow, preferably in an undifferentiated state. These cultures may be performed prior to or exclusive of transplantation of the HSC into a subject.

As will be apparent to one of ordinary skill in the art, the present invention will find application in a variety of compositions and methods.

### EQUIVALENTS

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

All references, patents and patent applications that are recited in this application are incorporated by reference herein in their entirety.

What is claimed is:

1. A method for transplanting cells in a subject comprising:
administering to a subject in need thereof hematopoietic stem cells and mesenchymal cells in numbers sufficient to populate at least one cell lineage in the subject.

2. The method of claim 1, wherein the hematopoietic stem cells are cord blood stem cells.

3. The method of claim 2, wherein the cord blood stem cells are partially purified from cord blood.

4. The method of claim 1, wherein the mesenchymal cells are mesenchymal stem cells.

5. The method of claim 1, 2, 3 or 4, wherein the mesenchymal cells are umbilical cord matrix cells.

6. The method of claim 1, wherein the hematopoietic stem cells are isolated.

7. The method of claim 1, wherein the mesenchymal cells are isolated.

8. The method of claim 1, wherein the hematopoietic stem cells and mesenchymal cells are administered intravenously.

9. The method of claim 1, wherein the hematopoietic stem cells and mesenchymal cells are administered locally.

10. The method of claim 1, wherein the subject has a disorder treatable by hematopoietic stem cell transplantation.

11. The method of claim 10, wherein the disorder is a hematopoietic deficiency or malignancy.

12. The method of claim 1, wherein the method results in more rapid engraftment compared to transplantation of the hematopoietic stem cells in the absence of the mesenchymal cells.

13. The method of claim 1, wherein the method results in higher engraftment levels compared to transplantation of the hematopoietic stem cells in the absence of the mesenchymal cells.

14. The method of claim 1, wherein the number of hematopoietic stem cells administered is below a number required to repopulate at least one lineage in the subject in the absence of the mesenchymal cells.

15. The method of claim 1, wherein the hematopoietic stem cells and mesenchymal cells are combined before administration to the subject.

16. The method of claim 1, wherein the mesenchymal cells are administered before, during and after the hematopoietic stem cells.

17. The method of claim 1, wherein the mesenchymal cells are administered during and after the hematopoietic stem cells.

18. The method of claim 1, wherein the mesenchymal cells are administered after the hematopoietic stem cells.

19. The method of claim 1, wherein the mesenchymal cells are administered daily, weekly, biweekly, or monthly.

20. The method of claim 1, further comprising administering a therapeutic agent to the subject.

21. A method for improving the outcome of a hematopoietic stem cell transplant in a subject comprising:
administering to a subject in need thereof hematopoietic stem cells and mesenchymal cells in an amount sufficient to improve the outcome of the hematopoietic stem cell transplant in the subject as compared to the outcome in the absence of the mesenchymal cells.

22. A pharmaceutical composition comprising:
isolated hematopoietic stem cells and isolated mesenchymal cells formulated in a pharmaceutically acceptable carrier,
for use in treating a subject having a disorder that benefits from populating at least one cell lineage.

23. The composition of claim 22, wherein the hematopoietic stem cells are cord blood stem cells.

24. The composition of claim 22, wherein the mesenchymal cells are mesenchymal stem cells.

25. The composition of claim 24, wherein the mesenchymal cells are umbilical cord matrix cells.

26. The composition of any one of claim 23-25, wherein the disorder is selected from the group consisting of
myelodysplasia, aplastic anemia, Fanconi's anemia, Sickle cell disease, Diamond Blackfan anemia, Schachman Diamond disorder, thalassemia, acute lymphocytic leukemia (ALL), acute myelogenous (myeloid) leukemia (AML), chronic lymphocytic leukemia (CLL), B-cell chronic lymphocytic leukemia, chronic myelogenous leukemia (CML), juvenile chronic myelogenous leukemia (JCML), juvenile myelomonocytic leukemia (JMML), T cell lymphoma, B cell lymphoma, Hodgkin's lymphoma, non-Hodgkin's lymphoma, severe combined immunodeficiency disease (SCID), breast cancer, renal cell (kidney) cancer, ovarian cancer, pancreatic cancer, small-cell lung cancer, melanoma, neuroblastoma, pediatric sarcoma, amyloidosis, diffuse progressive systemic sclerosis, alpha-L-iduronidase deficiency syndrome (Hurler's syndrome or disease), osteopetrosis, Gaucher's disease, globoid cell leukodystrophy, and adrenoleukodystrophy.

27. A use of isolated hematopoietic stem cells and isolated mesenchymal cells for the manufacture of a pharmaceutical preparation for treating a disorder that benefits from populating at least one cell lineage.

28. The use of claim 27, wherein the hematopoietic stem cells are cord blood stem cells.

29. The use of claim 27, wherein the mesenchymal cells are mesenchymal stem cells.

30. The use of claim 29, wherein the mesenchymal cells are umbilical cord matrix cells.

31. The use of any one of claim 27-30, wherein the disorder is selected from the group consisting of
myelodysplasia, aplastic anemia, Fanconi's anemia, Sickle cell disease, Diamond Blackfan anemia, Schachman Diamond disorder, thalassemia, acute lymphocytic leukemia (ALL), acute myelogenous (myeloid) leukemia (AML), chronic lymphocytic leukemia (CLL), B-cell chronic lymphocytic leukemia, chronic myelogenous leukemia (CML), juvenile chronic myelogenous leukemia (JCML), juvenile myelomonocytic leukemia (JMML), T cell lymphoma, B cell lymphoma, Hodgkin's lymphoma, non-Hodgkin's lymphoma, severe combined immunodeficiency disease (SCID), breast cancer, renal cell (kidney) cancer, ovarian cancer, pancreatic cancer, small-cell lung cancer, melanoma, neuroblastoma, pediatric sarcoma, amyloidosis, diffuse progressive systemic sclerosis, alpha-L-iduronidase deficiency syndrome (Hurler's syndrome or disease), osteopetrosis, Gaucher's disease, globoid cell leukodystrophy, and adrenoleukodystrophy.
